(19) United States
(12) Patent Application Publication
(10) Pub. No.: US 2009/0208543 A1
(43) Pub. Date: Aug. 20, 2009

(54) METHOD AND APPARATUS FOR APPLYING A PROTECTIVE ORAL CARE COMPOSITION

(75) Inventor: Salim A. NATHOO, Piscataway, NJ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Oral Health Clinical Services, Piscataway, NJ (US)

(21) Appl. No.: 12/357,924

(22) Filed: Jan. 22, 2009

Related U.S. Application Data
(60) Provisional application No. 61/022,659, filed on Jan. 22, 2008.

(51) Int. Cl. A61K 8/02; A61K 8/18; A61K 8/22; A61P 1/02

(52) U.S. Cl. 424/401; 424/49; 424/55

(57) ABSTRACT

Compositions and methods for whitening human teeth containing peroxide and a hydroperoxidase inhibitor. The compositions exhibit superior teeth whitening effects while also retaining a greater amount of peroxide compared to compositions not containing the hydroperoxidase inhibitors. The compositions may be formulated in a hydrocarbon humectant free vehicle comprising a peroxide compound, a hydroperoxidase inhibitor, a chelating agent, an anti-oxidizing agent, and a thickening agent. The compositions may further include compounds to reduce oxidative stress. Also disclosed is a delivery system including a flexible film, an oral care composition that contacts the teeth, an adhesive material that generally contacts the soft tissues, and a physical barrier that prevents contact between the oral care composition and the adhesive material.

### CROSS-REFERENCES TO RELATED APPLICATIONS

This application claims priority to and benefit under 35 U.S.C. § 119(e) to U.S. Provisional Appln. No. 61/022,659, filed Jan. 22, 2008, the disclosure of which is herein expressly incorporated by reference in its entirety.

### BACKGROUND OF THE INVENTION

The invention relates generally to compositions and methods for whitening human teeth and reducing oxidative stress. The invention also relates to a delivery system that includes applying a flexible film comprising an oral composition and an adhesive to the teeth. The flexible film prevents the oral composition and the adhesive from mixing.

### RELATED ART

A tooth is comprised of an inner dentin layer and an outer hard enamel layer that is coated with a protective layer called the pellicle. Teeth may generally become stained when chromogenic materials penetrate the enamel or get trapped within the pellicle. A tooth stain classification system, termed the “N” or “Nathoo Classification System,” has been proposed.

One type of direct dental stain is an N1 type stain that occurs when a chromogenic material binds to the tooth surface to cause discoloration similar in color to that of the unbound chromogen. Another type of direct dental stain is the N2 type stain, in which a chromogenic material binds to the tooth surface and subsequently undergoes a color change after binding to the tooth. An N3 stain is an indirect dental stain, caused by the binding of a colorless material (prechromogen) to the tooth, whereby a chemical reaction converts the prechromogen into a chromogen thereby causing the tooth stain.

Tooth stains may be either extrinsic or intrinsic, depending upon their location within the tooth structure. For example, extrinsic staining of the acquired pellicle arises when compounds such as tannins and other polyphenolic compounds become trapped in and tightly bound to the proteinaceous layer on the surface of the teeth. Naturally occurring tooth stains may also be caused by the adherence of bacteria and bacterial by-products to the tooth surface. Bacterial enzymes such as esterases may also cause dental stain by breaking down the natural constituents of the oral cavity such as matrix proteins which subsequently adhere to the tooth surface. Chemical reactions that occur naturally on the tooth surface involving amino acids and sugars can also induce dental stains by the Maillard reaction.

Extrinsic dental stains may usually be removed by mechanical methods such as by using toothbrushes and toothpastes that remove all or part of the acquired pellicle together with the associated stain. In contrast, intrinsic staining occurs when chromogens or prechromogens penetrate the enamel and the dentin and become entrapped or bound within the tooth structure. Intrinsic staining may also arise from systemic sources of chromogens or prechromogens. For example, administration of tetracycline during enamel development may lead to intrinsic staining. This type of stain is not amenable to mechanical methods of tooth cleaning and generally requires the use of chemicals such as hydrogen peroxide that can penetrate into the tooth structure to decolorize the chromogenic material.

The type of stain removal method depends upon individual requirements. For example, individuals desiring rapid whitening of teeth can usually have it done in the dental office. One method for whitening teeth used by dental professionals involves the use of a 30% to 35% hydrogen peroxide solution used in combination with heat and/or light to promote an oxidation reaction. Although this method produces quick results, it is expensive, and may require external activation of the bleach, thus confining the patient in a fixed position to the dental chair for extended periods of time.

Another professional method for bleaching teeth involves the use of hydrogen peroxide generating compounds such as urea peroxide (carbamide peroxide) at concentrations of 10%-20%. Urea peroxide rapidly breaks down into hydrogen peroxide due to the water present in saliva. This method known as at-home use dentist dispensed bleaching system involves the use of a mouth guard or tray within which the bleaching agent is placed. The tray is then placed upon the patient's teeth and bleaching ensues. This method of treatment has disadvantages including visits to the dental office for fabrication of a mouth guard, extended wear time of the splint, tooth sensitivity, and irritation of oral tissues.

The disadvantages associated with teeth bleaching products that include oxygen liberating bleaching compounds is the tendency of these products to decompose within a relatively short period of time following manufacture or usage with concomitant loss of all or a substantial amount of the available oxygen, thereby limiting the efficacy of these products as teeth whitening compositions.

Peroxide instability issues following manufacture have been addressed by utilizing anhydrous powders or water-free pastes or gels. However, disadvantages associated with these products are that, due to the absence of water, application of the oral composition tends to desiccate oral tissue, which leads to irritation and tissue damage. In order to overcome these disadvantages, oxygen releasing toothpaste compositions which have greater water content have been developed.

The compositions described above all have the primary disadvantage of having limited stain removal or tooth whitening effectiveness. A number of mechanisms may be responsible for the observed loss of peroxide during the teeth bleaching process. This includes the dilution of the bleaching agent by saliva. The problem of excessive dilution by saliva has been addressed by the use of thick/viscous compositions. A number of the compositions described above also contain metal chelating agents which address the issue of peroxide stability during manufacturing and storage but not the issue of peroxide stability during the bleaching process.

It is well known that enzymes known as hydroperoxidases are present in the oral cavity. These enzymes, which include catalyses, are responsible for the catalytic decomposition of peroxides into oxygen and water. Both oxygen and water have been shown to have a limited effect upon bleaching teeth. Thus, there is a need for bleaching compositions in which the degradation of peroxide is prevented, inhibited or mitigated.

The advantages of inhibiting hydroperoxidases, and particularly catalyses, have been illustrated in U.S. Pat. No. 5,885,412, which discloses that less peroxide is required for bleaching cellulose paper fibers in the presence of catalase inhibitors, such as sodium fluoride, sodium chloride, sodium bromide, hydroxylamine, sulfides and reducing agents e.g., ascorbic acid. Additionally, U.S. Pat. No. 3,606,990 discloses the utility of using hydroperoxidase inhibitors in preventing premature decomposition of peroxides in bleaching and washing of laundry.

In addition to using catalase inhibitors to prevent decomposition of peroxides, various methods have been developed to effectively apply bleaching agents to the teeth. It is generally recognized that in order to increase the teeth whitening effectiveness of various compositions, the contact time between the teeth surfaces and the peroxide formulation may be increased. For example, U.S. Pat. No. 6,096,328 describes a system for delivering an oral care substance to the oral surface that includes a strip of flexible material that forms to the contours of the oral surface. The oral care substance is applied to the flexible strip to contact the oral surface. The oral care substance also provides adhesive attachment between the strip and the oral surface to hold the delivery system in place for a sufficient amount of time to allow the active agent to act upon the oral surface.

The delivery system described in U.S. Pat. No. 6,096,328 also contains shallow pockets to hold the oral care substance, such as a peroxide preparation, and to prevent contact between saliva and the oral care substance. A teeth whitening system based upon this patent is available commercially under the trademark CREST WHITE STRIPS®.

The disadvantages of using the strips are similar to those of tray products. In addition, clinical studies have shown that the incidence of gingival irritation in users of CREST WHITE STRIPS® is approximately 40%. The irritation may be due to excessive contact between the peroxide preparation with the oral tissues. The strips can also be easily dislodged as a result of normal activities such talking, sleeping etc. The dislodgment may occur as a result of penetration of saliva and/or as a result of production of oxygen occurring as a result of peroxide degradation and increased gaseous pressure on the side of the strip in contact with the teeth tending to deform the strip and aid in the dislodgment. To overcome some of the disadvantages described above, U.S. Pat. No. 6,746,679 describes the use of bioadhesive materials to isolate a bleaching composition and protect the tissues of the oral cavity.

U.S. Pat. No. 6,860,736 attempts to overcome the disadvantages of using strips of material as described in U.S. Pat. No. 6,096,328 by using a dental tray, a horseshoe, a “u” shaped or a “v” shaped delivery system with an auxiliary adhesive barrier composition. The purpose of the adhesive barrier layer is to protect the oral care composition from saliva or moisture, maintain contact with the teeth, prevent contact with the soft tissue, and minimize diffusion of the composition into the user's oral cavity.

However, there are several problems associated with the delivery system of U.S. Pat. No. 6,860,736. First, there is no physical barrier to separate the adhesive barrier layer from the oral care composition. Thus, the adhesive barrier and the oral care composition may mix, thus reducing the uniformity of the concentration of the oral care composition upon transportation and usage. Further, when using reactive materials such as peroxide preparations, chemical interactions and reactions may occur between the peroxide and the adhesive composition. The above disclosure also lacks the ability for gases, produced as a result peroxide degradation, to escape thus, causing increased pressure and dislodgment of the delivery system. In addition, the above mentioned invention lacks indentations or shallow pockets to provide reservoirs for the oral care composition. Preformed dental trays such as horseshoes, “u” shaped or “v” shaped delivery systems also tend to be bulky and require proper placement over the teeth and are thus are considered to be difficult to use. Yet another disadvantage is that studies have shown that most of the hydrogen peroxide in the splint or tray system is lost within 30 to 50 minutes.

In order to overcome the disadvantages associated with the tray or the strip type of materials, newer “paint on” products have become available. An example of such a product is SIMPLY WHITES® (Colgate-Palmolive Co.). However, other studies have shown that in the “paint on” products, the majority of peroxide is lost within 5 minutes of product application.

While the use of hydroperoxidase inhibitors has been shown to be effective, there is a need to further prevent the decomposition of peroxide bleaching agents upon introduction the oral cavity to provide a better bleaching effect. Accordingly, it is a feature of the invention to provide an oral care composition that comprises a hydroperoxidase inhibitor to prevent rapid decomposition of peroxide during bleaching or stain removal processes resulting in a more rapid teeth whitening effect.

Without being bound to a specific theory, it is believed that the rapid decomposition of peroxide tends to release excessive amounts of free radicals, thus, increasing the oxidative stress in the oral cavity. Some products currently available on the market are said to produce hydroxyl radicals as a result of a photo-fenton reaction between iron and peroxide (Dental Products Report, December 2006, Trends in Dentistry Issue, page 102). However, it has been reported that the hydroxyl radicals are one of the most reactive and damaging free radical species (Battino et al, Crit. Rev Oral Biol Med 10:458-476, 1999). According to the same authors, the type and extent of the damage at the site of generation of a harmful species, such as hydroxyl radicals, could lead to severe purine or pyrmidine modification or to DNA strand breakage if it take place close to the DNA, or no biological consequences if it occurs close to a generic enzyme molecule whose concentration is not critical. Currently the consequences of oxidative stress and free radical formation are not clear. However, a study presented at the 6^{th} International Conference on Head and Neck cancer in 2004 suggested a link between Oral Cancer and teeth bleaching. A study presented at the International Association of Dental Research Meeting in 1999 also suggested a link between cancer progression when hydrogen peroxide is used in combination with a cancer promoter DMBA (Boyd et al, Journal of Dental Research, 1999 Abstract 2641). The results of the above study, however, were later retracted. More recently, an in-vitro study showed that dental bleaching compounds contributed to DNA damage (Ribeiro et al, Braz Oral Res. 20:47-51, 2006). Accordingly it is a feature of this invention to provide compositions and a delivery system to enhance the safety of the teeth bleaching process and alleviate possible concerns regarding the use of peroxide containing products.

Other studies have shown that teeth bleaching agents release mercury from dental amalgam (Certosimo et al, General Dentistry, July-August 2003, pages 356-359). Accordingly it also is a feature of this invention to provide compositions and a delivery system to minimize mercury release.

There is also a need to provide a delivery system in which an adhesive material does not react with the bleaching composition. In order to avoid a reaction between the adhesive material and the bleaching composition, the invention provides a delivery system in which a physical barrier is used to separate the bleaching composition and the adhesive. This delivery system also allows the adhesive material to adhere to the oral soft tissues, thus avoiding soft tissue irritation, leakage of active ingredients such as peroxide into the oral cavity and preventing the dislodgement of the delivery system. The adhesive system would preferably be sufficiently porous to allow for gases to escape yet contain ingredients that would tend to counteract the negative effects of peroxide and have beneficial effects upon ones oral cavity and general health.

### SUMMARY OF THE INVENTION

The invention satisfies the above needs by forming tooth whitening or tooth bleaching compositions that contain one or more catalase inhibitors formulated in a vehicle comprising a peroxide compound, a metal chelating agent, water, a thickening agent, and an anti-oxidant. The compositions of the invention may be substantially free of hydrocarbon humectants. As used herein, the term “hydrocarbon humectant” refers to a high boiling point carbon, hydrogen and oxygen containing compound that may be used to prevent the oral care composition from hardening upon exposure to air. Examples hydrocarbon humectants may include glycerin, sorbitol, polyethylene glycol, propylene glycol and the like.

In an embodiment of the invention, the oral compositions may be in the form of a gel or a mouth rinse that exhibits superior teeth whitening effects when compared to compositions that do not contain hydroperoxidase inhibitors. Moreover, the compositions of the invention may be used in the dental office and also for in-home use such as brushing, rinsing, sequential application e.g., as pre brush or post brush applications, pre and post professional teeth whitening applications, splint applications, paint on applications and application onto tooth surfaces using strips of polymeric materials.

Another embodiment of the invention provides a delivery system in which a physical barrier may be placed between an adhesive and an oral care composition on a flexible strip to separate and prevent a reaction between the adhesive material and the bleaching and/or dental composition.

In an embodiment of the invention, a method for preparing an oral care composition includes preparing a flexible film, applying an oral care composition to the flexible film, applying an adhesive material to the flexible film, and forming a physical barrier that prevents contact between the oral composition and the adhesive material. The flexible film may be less than about 3 mm thick. The flexible film may includes polymers, natural and synthetic woven materials, non-woven material, foil, paper, rubber, and combinations thereof. The oral care composition may include a peroxide compound, a hydroperoxidase inhibitor, a chelating agent, an anti-oxidizing agent, and a thickening agent, and the oral care composition may be in an orally acceptable vehicle. The adhesive material may include natural gums, synthetic polymeric gums, alkyl vinyl ether-maleic acid copolymers, synthetic polymers, mucoadhesive polymers, hydrophilic polymers, saccharide derivatives, cellulose derivatives, and adhesive materials commonly employed in denture stabilizing compositions and compatible with the subject flexible film of the present invention, and mixtures thereof. The physical barrier may be an indentation or a raised region of the flexible film, a dividing strip that is placed or bonded to the flexible film, a T-shaped barrier, an orthodontic tube, or an orthodontic sleeve with a wire inserted in the orthodontic sleeve. The wire may include metal wires including copper wire, stainless steel wires, and aluminum wires; polymeric wires; and thermal arch wires. The method may further include forming an orthodontic wire along a length of the flexible film or along a length and a width of the flexible film. A laminate including a mesh may be formed in between two flexible films.

In another embodiment of the invention, a delivery system for an oral care composition includes a flexible film, an oral care composition, an adhesive material and a physical barrier that prevents contact between the oral composition and the adhesive material. The flexible film may be less than about 3 mm thick. The flexible film may include polymers, natural and synthetic woven materials, non-woven material, foil, paper, rubber, and combinations thereof. The oral care composition may include a peroxide compound, a hydroperoxidase inhibitor, a chelating agent, an anti-oxidizing agent, and a thickening agent, and the composition may be in an orally acceptable vehicle. The adhesive material may include natural gums, synthetic polymeric gums, alkyl vinyl ether-maleic acid copolymers, synthetic polymers, mucoadhesive polymers, hydrophilic polymers, saccharide derivatives, cellulose derivatives, and adhesive materials commonly employed in denture stabilizing compositions and compatible with the subject flexible film of the present invention, and mixtures thereof. The physical barrier may be an indentation or a raised region of the flexible film, dividing strips that are placed or bonded to the flexible film, a T-shaped barrier, an orthodontic tube, or an orthodontic sleeve with a wire inserted in the orthodontic sleeve. The wire may include metal wires including copper wire, stainless steel wires, and aluminum wires; polymeric wires; and thermal arch wires. The orthodontic wire may be formed along a length of the flexible film or along a length and a width of the flexible film. In addition, a laminate including a mesh may be formed in between two flexible films.

In another embodiment of the invention, the oral care composition is a tooth bleaching composition and includes a peroxide compound, a hydroperoxidase inhibitor, a chelating agent, an anti-oxidizing agent, and a thickening agent, and the composition is in an orally acceptable vehicle. The peroxide compound may include an oxidizing agent present in a concentration in a range of about 1% to about 50% by weight of the composition. The oxidizing agent may include hydrogen peroxide, urea peroxide, glyceryl peroxide, benzoyl peroxide, PVP-hydrogen peroxide, sodium peroxide, sodium percarbonate, sodium perborate, calcium peroxide, and combinations thereof. The hydroperoxidase inhibitor may be a catalase. The hydroperoxidase inhibitor may be selected from the group consisting of sodium acetate, a halogen releasing compound, resorcinol, and combinations thereof. The hydroperoxidase inhibitor is typically present in a concentration in a range of about 0.01% to about 20% by weight of the composition. The thickening agent may include polymeric compounds with hydrophilic and hydrophobic groups. For example, the thickening agent may be a polyoxyethylene polyoxypropylene block copolymer with a concentration in a range of about 15% to about 50% by weight of the composition. The thickening agent may also be carboxypolymethylene present in a concentration in a range of about 0.5% to about 10% by weight. The thickening agent may also include a combination of a polyoxyethylene polyoxypropylene block copolymer and carboxypolymethylene. The chelating agent may include one or more of a condensed phosphate chelating agent group, an organic chelating agent group, and a metal precipitating group. The condensed phosphate metal chelating agent may be sodium or potassium pyrophosphates, tripolyphosphate, hexametaphosphate, and combinations thereof. The organic metal chelating agent may include phosphoric acid, xylitol, glycerophosphoric acid, gluconolactone, phosphonic acid, di- and tri-phosphonic acid compounds, etidronic acid, edetic acid, disodium ethylenediamine tetraacetate, trisodium ethylenediamine tetraacetate, amino polycarboxylic acids, and combinations thereof. The metal precipitating agent may include sodium fluoride, sodium monofluorophosphate, citric acid and its salts, triethylcitrate, and combinations thereof. The chelating agent may have a concentration in a range of about 0.5% to about 5% by weight of the composition. The anti-oxidizing agent may include a peroxide stabilizing compound in a concentration in a range of about 0.1% to about 5% by weight of the composition. The anti-oxidizing agent may be triethylcitrate, for example. The concentration of the anti-oxidizing agent may be about 0.002% to about 20%. The tooth bleaching composition may further include a surface active agent such as non-ionic detergents, anionic detergents, and cationic detergents. The concentration of the surface active agent may be about 0.01% to about 8% by weight of the composition. The surface active agent may be sodium lauryl sulfate with a concentration in a range of about 0.01% to about 5% by weight of the composition. The tooth bleaching composition may further include a carrier that is substantially free of a hydrocarbon humectant such as water. The tooth bleaching composition may further include a pH value adjusting agent that adjusts the composition to a pH of about 3 to about 7. The pH adjusting agent may be sodium acetate or a catalase inhibitor.

Additional features, advantages, and embodiments of the invention may be set forth or apparent from consideration of the following detailed description, drawings and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 is a top view of the delivery system according to an embodiment of the invention.

FIG. 2 is a cross-sectional view of the delivery system of the invention as shown in FIG. 1.

FIG. 3 is a cross-sectional view according to other embodiment of the invention.

FIG. 4 is a cross-sectional view according to other embodiment of the invention.

FIG. 5A is a cross-sectional view of the delivery system according to another embodiment of the invention.

FIG. 5B is a front view of the delivery system shown in FIG. 5A as applied to teeth.

FIG. 6 is a front view of a delivery system according to another embodiment of the invention, as applied to teeth.

FIG. 7A is a cross-sectional view of the delivery system according to another embodiment of the invention.

FIG. 7B is a cross-sectional view of the delivery system according to another embodiment of the invention.

FIG. 8A is a top view of a delivery system according to another embodiment of the invention, as described in Example 1.

FIG. 8B is a top view of a delivery system according to another embodiment of the invention, as described in Example 2.

### DETAILED DESCRIPTION OF THE INVENTION

It is understood that the invention is not limited to the particular methodology, protocols, devices, apparatus, materials, and reagents, etc., described herein, as these may vary. It is also to be understood that the terminology used herein is used for the purpose of describing particular embodiments only, and is not intended to limit the scope of the invention. It must be noted that as used herein and in the appended claims, the singular forms “a,” “an,” and “the” include plural reference unless the context clearly dictates otherwise. Thus, for example, a reference to “a catalase inhibitor” is a reference to one or more catalase inhibitors and equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the invention. All references cited herein are incorporated by reference herein in their entirety.

According to an embodiment of the invention, the dental bleaching composition may comprise a peroxide compound, a hydroperoxidase inhibitor, a metal chelating agent, an anti-oxidation agent and a thickening agent. As used herein, the term hydroperoxidases e.g., “catalases” refer to catalysts that may be found in the oral cavity and promote the decomposition of peroxides into oxygen and water.

Suitable peroxide compounds used to prepare the oral compositions of the invention may include hydrogen peroxide, organic peroxides including urea peroxide, glyceryl peroxide, benzoyl peroxide, PVP-hydrogen peroxide and the like, metal containing peroxides including sodium peroxide, calcium peroxide, sodium perborate, sodium percarbonate and the like. In particular, the peroxides may be hydrogen peroxide and urea peroxide, which may be used individually or in any combination.

Typically, the composition of the invention comprises at least about 0.1% by weight of the peroxide. Specifically, the peroxide compound may be present in a concentration ranging from about 1% to about 50% by weight of the composition. In particular, the concentration of the peroxide may be in a range of about 3% to about 35% by weight of the composition. A typical peroxide concentration in the composition is generally in a range of about 2% to 10% by weight for home use products and in a range of about 10% to 50% for dental professional use.

The composition of the invention also comprises compounds that retard the decomposition of peroxides by inhibiting the activity of hydroperoxidases or catalases. These hydroperoxidase inhibitors may include compounds that release halogens such as alkali metal halogenated compounds such as sodium fluoride, sodium chloride, hydrofluoric acid, hydrochloric acid, potassium fluoride, cuprous fluoride, tin fluorides such as stannous fluoride or stannous chlorofluoride, sodium fluorosilicate, ammonium fluorosilicate, sodium monofluorophosphate, alumina mono- and di-fluorophosphate.

Other catalase inhibiting compounds may include, but are not limited to, sulfides, aminoguanadine, hydroxylamine, hydroxylammonium chloride, sodium hypochlorite and other chlorine bleaches or chlorine releasing compounds such as calcium hypochlorite, sodium acetate and reducing agents such as ascorbic acid, and dithiothretrol. Fluorine containing compounds may also be used because they have the additional benefits of preventing dental caries and may enhance the storage stability of peroxide compositions. Thus, the catalase inhibiting compounds may include fluorinated compounds and orally acceptable compounds such as sodium acetate and resorcinol. In addition, catalase inhibitors described in U.S. Pat. Nos. 3,606,990, 5,885,412 and 5,424,203, which are incorporated herein by reference, may also be included in the composition.

In an embodiment, the composition of the invention may comprise the catalase inhibiting compound at a concentration in a range of about 0.01% to about 20% by weight of the composition, and specifically in the range of about 0.05% to about 10% by weight of the composition.

In an embodiment of the invention, thickening or gelling agents used in the formulation of the bleaching composition may include nonionic polyoxyethylene polyoxypropylene block copolymers and polymers of acrylic acids and polyacrylates. Representative examples of polyoxyethylene polyoxypropylene block copolymers may include block copolymers having the formula HO(C₂H₄O)_{b}(C₃H₆O₆)ₐ(C₂H₄O)_{b}H, wherein a may be an integer such that the hydrophobic base represented by (C₃H₆O₆) has a molecular weight in the range of about 2750 to about 4000, and b may be an integer such that the hydrophilic portion (moiety) represented by (C₂H₄₀) constitutes about 70% to about 80% by weight of the copolymer. Block copolymers of this composition are commercially available, including PLURONIC F® (BASF).

Suitable polyacrylates may include compounds such as carboxypolymethylene which is a slightly acidic vinyl polymer with active carboxyl groups. For example, CARBOPOL® (B. F. Goodrich) may be used.

The concentration of the thickening agent may vary with the type of thickening agent and can be used alone or in combination. Polyoxyethylene polyoxypropylene block copolymers may be used at a concentration ranging from about 15% to about 50% by weight of the composition, and in particular in a range of about 20% to about 35%. In a specific embodiment, the concentration of carboxypolymethylene used may be in a range of about 0.5% to about 10%, and specifically in a range of about 1% to about 5% by weight of the composition.

Since carboxypolymethylene is a polycarboxylic acid, it tends to excessively lower the pH of the resulting bleaching composition. The pH can be adjusted from about pH 3 to about pH 7 by adding a pH adjusting agent, such as a base, to the composition. Inorganic and organic bases or a combination thereof may be used and may include bases such as sodium hydroxide, potassium hydroxide, ammonium hydroxide, sodium borate, triethanolamine and other amino compounds such as amino acids. Certain bases such as sodium acetate may also play a role in the inhibition of catalase. In particular, the pH of the composition may be in a range of about pH 2 to about pH 9 and more particularly, in a range of about pH 3 to about pH 7.

The tooth bleaching composition of the invention further includes agents that chelate metal ions. The chelating agents may comprise a blend of metal solubilizing agents and metal precipitating agents. The metal solubilizing agents may include a condensed pyrophosphate compound. For purposes of this invention “condensed phosphate”, as used herein, relates to an inorganic phosphate composition containing two or more phosphate species in a linear or cyclic pyrophosphate form. In particular, a condensed phosphate may be sodium or potassium pyrophosphate but may also include tripolyphosphate, hexametaphosphate, cyclic condensed phosphate or other similar phosphates that are well known in the art.

The blend may also include an chelating agent. As used herein, the term “phosphate” may include phosphoric acid, xylitol, triethyl citrate, glycerophosphoric acid, gluconolactone, phosphonic acid, di- and tri phosphonic acid compound or its salts. For example, the phosphonic acids may include DEQUEST 2010® (Monsanto) or 1-hydroxyethylidene-1,1-diphosphonic acid, which is also known as Etidronic acid. Other examples of organic chelating agents may include edetic acid and its salts, such as disodium ethylenediamine tetraacetate, trisodium ethylenediamine tetraacetate and other amino polycarboxylic acids.

The blend may also include a metal precipitating chelating agent. As used herein, the term “precipitating chelating agent” refers to an agent that binds to metals and causes the metal to precipitate and may include halogens such as sodium fluoride, sodium monofluorophosphate, citric acid and its salts such as sodium citrate, potassium citrate, oxalic acid and its salts such as sodium oxalate. At least one chelating agent from each group i.e., organic chelating agent, condensed phosphate chelating agent, and metal precipitating agent may be incorporated into the composition of the invention. The chelating agents may be incorporated in the oral care compositions of the invention in an amount within the range of about 0.1% to about 10.0% by weight and specifically in the range of about 0.5% to about 5.0% by weight of the composition.

The compositions of the invention may further comprise an antioxidant or mixtures thereof. The purpose of the antioxidant is to retard the auto-oxidation of the peroxide compound and/or quench free radicals, thus reducing production of unwanted gases and increasing the safety profile. Suitable antioxidants for use herein may include, but are not limited to organic acids like ascorbic acid, salicyclic acid, compounds derived from salicyclic acid, such as acetyl salicyclic acid and alike, adipic acid, tartaric acid, omega 3 fatty acids, esters of omega 3-fatty acids, unsaturated fatty acids ubiquniones, such as coenzyme q, sorbic acid, amines such as lecithin, or amino acids such as glutamine, methionine and cysteine, or esters such as ascorbil palmitate, ascorbil stearate, n-acetyl cysteine and triethylcitrate, other compounds such as selenium, amino-acid selenium compounds such as methionine—selenium and like or mixtures thereof. In particular, the antioxidant may be triethylcitrate (ethyl citrate or triethyl 2-hydroxy-1,2,3-propanetricarboxylate) because, in addition to having anti-oxidation properties, this compound is extensively used in cosmetic products as a sequestrant, an antimicrobial agent and an inhibitor of esterases. Further, triethyl citrate may be used in the filters of cigarettes to absorb odoriferous materials from tobacco smoke. Hence, the compositions described herein may also be useful to prevent halitosis.

The compositions herein may comprise up to about 10% by weight of the total composition of an antioxidant or mixtures thereof, and specifically in the range of about 0.002% to about 20%, more specifically in the range of about 0.01% to about 10%.

Surfactants may also be included in the bleaching compositions of the invention. These agents may serve as solubilizing, dispersing, emulsifying agents, or as an agent that reduces the surface tension of the teeth in order to increase the contact area between the tooth and the peroxide. The surfactants may also help solubilize, disperse, or emulsify the stain within the intercrystalline spaces, thus further aiding the penetration of peroxide. Particularly useful surfactants may include nonionic surfactants such as a water soluble polyoxyethylene monoester of sorbitol with a C₁₀₋₁₈ fatty acid ester of sorbitol (and sorbitol anhydrides), consisting predominantly of the monoester, condensed with about 10 to about 30, and, specifically, about 20, moles of ethyleneoxide. The fatty acid (aliphatic hydrocarbon-monocarboxylic acid) may be saturated or unsaturated, e.g. lauric, palmitic, stearic, or oleic acids. Anionic surfactants may be used, such as water soluble salts of higher fatty acid monoglyceride monosulfates, such as sodium salts of the monosulfated monoglycerides, or hydrogenated coconut oil fatty acids and higher alkylsulfates, such as sodium lauryl sulfate and alkyl aryl sulfonates, such as sodium dodecyl benzene sulfonate. Other surfactants such as fluorinated surfactants and surface tension reducing materials may also be incorporated within the compositions.

The surfactant may be present in a concentration in a range of about 0.01% to about 8.0% by weight, and specifically, in a range of about 0.05% to about 5% by weight of the oral composition.

In a further embodiment, the carrier of the composition may comprise water and may be substantially free of hydrocarbon humectants. One purpose for the humectants in a dentifrice formulation is to prevent the composition from premature desiccation upon repeated opening and closing of a container containing the dental product e.g. a tooth paste tube. Thus, for a multiple use product, it may be desirable to include the humectants in the composition. However, the compositions may be substantially free of hydrocarbon humectants.

The following examples are further illustrative of the invention, but it is understood that the invention is not limited thereto. Other suitable ingredients found in oral care products such as abrasives, flavors, caries, control agents, hypersensitive agents, alcohols and sweeteners can also be added to the inventive compositions. All amounts and proportions referred to herein and in the appended claims are by weight unless otherwise indicated.

### EXAMPLES

### Specific Example 1

Gels were prepared as outlined in Table 1.

TABLE 1

| Ingredient % w/w | Ex 1 | Ex 2 | Ex 3 | Ex 4 | Ex 5 | Ex 6 |
|---|---|---|---|---|---|---|
| PLURONIC F127 | 30 | 30 | 30 | 30 | 30 | 30 |
| Water | 49.2 | 58.7 | 58.9 | 59.0 | 59.0 | 59.0 |
| Hydrogen peroxide (30%) | 10 | 10 | 10 | 10 | 10 | 10 |
| Polyethylene glycol 600 | 10 | — | — | — | — | — |
| Etidronic Acid | 0.2 | 0.2 | 0.2 | 0.2 | 0.2 | 0.2 |
| Sodium Lauryl sulfate | 0.2 | 0.2 | 0.2 | 0.2 | 0.2 | 0.2 |
| Sodium Pyrophosphate | 0.2 | 0.2 | 0.2 | 0.2 | 0.2 | 0.2 |
| Triethyl Citrate | 0.2 | 0.2 | 0.2 | 0.2 | 0.2 | 0.2 |
| Resorcinol | — | — | 0.2 | — | — | — |
| Sodium Fluoride | — | — | — | 0.2 | — | — |
| Sodium Chloride | — | — | — | — | 0.2 | — |
| Sodium Acetate | — | — | — | — | — | 0.2 |

The dental gels were prepared by adding etidronic acid, triethyl citrate and either resorcinol, sodium fluoride, sodium chloride or sodium fluoride to distilled water until a clear solution was obtained. Sodium lauryl sulfate was then added to the mixture and stirring continued until the surfactant dissolved. The resulting mixture was then transferred to a stainless steel premier vacuum mixer and PLURONICF127® and polyethylene glycol was added. The mixture was then mixed for 10 minutes without the vacuum, after this period the vacuum was turned on and mixing carried out for an additional 30 minutes. Hydrogen peroxide was then added and mixing continued under vacuum for an additional 30 minutes. The pH of all the compositions detailed above was then adjusted to pH 4 with sodium hydroxide.

The gels shown in Table 1 are described as ringing gels because they have a rigid jelly like consistency and when placed in a container and the sides tapped lightly, the gels maintain their original configuration.

Tooth bleaching effectiveness of the above gels was determined by using extracted human teeth. The teeth were freshly extracted, freed of all adherent tissues and stored in a sterile saline solution. The teeth were removed and the color was measured using a chroma meter commercially available from MINOLTA CR221. Readings were taken to record the L*, a*, and b* parameters. The teeth were then incubated in the various gels for 30 minutes. After this period, the gels were washed off and L*, a*, and b* parameters color re-measured. The change in color (Delta E) was then calculated using the CIE L*a*b* color difference equation:

Delta E=[(delta L*)²+(delta a*)²+(delta b*)²]^{0.5}

The results are shown in Table 2.

TABLE 2

| Tooth # | Example | Delta E (increase in whiteness) |
|---|---|---|
| 1 | Commercial Product | 3.28 |
| 2 | Ex 1 | 4.64 |
| 3 | Ex 2 | 4.92 |
| 4 | Ex 3 | 6.42 |
| 5 | Ex 4 | 5.32 |
| 6 | Ex 5 | 5.90 |
| 7 | Ex 6 | 6.29 |

Table 2 above shows the effect of catalase inhibitors upon whitening effectiveness. Tooth #1 was the commercial product, sold under the trade name of OPALESCENCE® (Ultradent Corporation). Ex 1 and Ex 2 lacked the catalase inhibitor. The carrier of peroxide in Ex 1 was a combination of water and polyethylene glycol whereas the carrier of peroxide in Ex 2 lacked the humectants and consisted exclusively of water. The results show that both Ex 1 and Ex 2 have a greater bleaching effect when compared with the commercial product. As detailed in U.S. patent application Ser. No. 09/920,674, the disclosure of which is expressly incorporated fully herein by reference in its entirety, the results also show that Ex 2 performs statistically better compared to Ex 1, showing that the humectant reduces bleaching activity. Further as detailed in the aforementioned application, Ex 3, 4, 5 and 6 contain the catalase inhibitor and show greater tooth whitening effectiveness when compared to compositions lacking the inhibitor.

When comparing all of the compositions comprising the inhibitors, sodium fluoride was found to be the least effective. Sodium fluoride may have reduced effectiveness because the fluoride ion may form insoluble complexes with the calcium present on the tooth surface or trace metal impurities present within the composition, thereby making it unavailable to prevent the catalytic activity of catalase. Further, as indicated above, insoluble fluorinated complexes may block enamel and dentinal tubules, thereby retarding the penetration of peroxide into the tooth structure.

In order to overcome this problem, dual component formulations may be prepared e.g., a dual chambered syringe or a dual chambered tube whereby the peroxide component is maintained in one chamber and the catalase inhibitor in the other chamber. The two components are then mixed manually or via the use of a static mixer and applied to the teeth. Alternatively, a catalase inhibitor can be applied to the teeth for a sufficient period of time to allow for the inhibition of catalase followed by application of the peroxide composition.

### Specific Example 2

As detailed in Table 3 below, compositions including CARBOPOL® were prepared to determine the clinical effect of a catalase inhibitor.

TABLE 3

| Ingredient % w/w | Ex A | Ex B | Ex C |
|---|---|---|---|
| CARBOPOL 940 | 2 | 2 | 2 |
| Water | 32.3 | 32.1 | 37.1 |
| Hydrogen peroxide (50%) | 60 | 60 | 60 |
| Polyethylene glycol 600 | 5 | 5 | — |
| Phosphoric Acid | 0.2 | 0.2 | 0.2 |
| Sodium Lauryl sulfate | 0.2 | 0.2 | 0.2 |
| Sodium Pyrophosphate | 0.2 | 0.2 | 0.2 |
| Triethyl Citrate | 0.1 | 0.1 | 0.1 |
| Resorcinol | — | 0.2 | 0.2 |

The dental gels were prepared by mixing CARBOPOL®, water and polyethylene glycol until a homogenous mixture was obtained. Then phosphoric acid, triethyl citrate and resorcinol were added to the sample and mixed. The mixture was then transferred to a double planetary mixer and hydrogen peroxide was added. The mixture was then mixed under vacuum for about 1 hour. The gel was then obtained by adding 6 g of triethanolamine and further mixing the sample under vacuum for an additional one hour. The final pH of the gel was adjusted to about pH 4 using sodium borate. Notably, the pH can also be adjusted with a base such as sodium acetate to obtain further catalase inhibition and may act as a replacement for resorcinol. Further, if desired, a flavoring agent can also be added to the composition.

An in vitro study using extracted human teeth was performed using the formulations Ex A, Ex B and Ex C, as described above in Table 3. A 35% solution of hydrogen peroxide purchased from Spectrum Chemical was used as a control. Notably, high concentration peroxide solutions have been and are extensively used for the purposes of bleaching teeth.

In the study described above, twelve freshly extracted human molars were used to examine the teeth whitening effectiveness. The shade of the teeth was measured using the VITA® Shade Guide commercially available from VITA before and after treatment with the above compositions. The change in color was determined by counting the difference in the number of VITA® Tabs before and after treatment. In this example, the teeth were randomly divided into four groups and incubated in the test solutions for one hour. After incubation the teeth were washed in water and the shade was measured. The results are shown in Table 5.

TABLE 5

| | Initial Shade | Final Shade | Shade Improvement |
|---|---|---|---|
| Ex A | A3 | B2 | 6 |
| Ex B | A3 | B1 | 8 |
| Ex C | A3 | B1+ | 9 |
| 35% Peroxide | A3 | A2 | 4 |

The data shown above shows that significant improvements can be obtained within one hour of using a higher concentration of peroxide in the inventive composition containing a catalase inhibitor. In Table 5 above, B1+ indicates a color lighter then the lightest shade on the VITA® shade guide. The data also show the greatest tooth whitening efficacy is obtained for the hydrocarbon free composition. Compositions which contained the humectant in addition to hydroperoxidase inhibitors and the anti-oxidant also gave a better whitening effect when compared to the commercial product.

### Specific Example 3

To further test the effects of a catalase inhibitor, a mouth rinse was prepared as shown in Table 6 below.

TABLE 6

| Ingredient | % (w/w) |
|---|---|
| Water (distilled) | 82.45 |
| Hydrogen Peroxide (35% Solution) | 9 |
| Pluronic F68 | 5 |
| Sodium Acetate (30% Solution) | 1.25 |
| Pluronic F127 | 1 |
| Phosphoric acid (80%) | 0.5 |
| Sodium Pyrophosphate | 0.4 |
| Polysorbate 20 | 0.2 |
| Sodium Lauryl Sulfate | 0.2 |
| Triethyl Citrate | 0.1 |
| Saccharin | 0.1 |

The above composition was prepared by adding sodium pyrophosphate to distilled water and allowing it dissolve by mixing. This was followed by adding PLURONIC F68 to the above mixture and allowing it to dissolve by mixing. Then, PLURONIC F127® was added and dissolved by mixing. Sodium acetate was then added and dissolved. Then, Polysorbate 20 was added and dissolved by mixing. Then, 0.005% Antifoam A was added to control excessive foaming. Sodium lauryl sulfate was then added to the mixture and allowed to dissolve by mixing. Then, phosphoric acid, triethyl citrate, and sodium saccharin were added and allowed to dissolve by mixing. Finally hydrogen peroxide was added and the composition was mixed.

To determine the effectiveness of the above rinse in whitening teeth, an in vitro study was performed as described above. To perform this study, twenty extracted teeth were cleansed and washed as described above. The color of the teeth was then measured using the MINOLTA CR221 CHROMA METER® and were then were randomly divided into two groups of 10 teeth. The teeth were then incubated for one hour in the rinse composition described in Table 6. A commercially available peroxide rinse, such as PEROXYL® (Colgate Oral Pharmaceuticals), was used as a control.

The change is color was determined by measuring the L*, a*, b*, parameters and using the color difference equation described above. The results are shown in Table 7 below:

TABLE 7

| Sample | n | ΔE |
|---|---|---|
| Rinse (Table 6) | 10 | 4.07 (2.58) |
| PEROXYL | 10 | 0.61 (2.24) |

Statistical analysis by the two tailed t-test showed that the inventive rinse is significantly more effective at whitening teeth when compared to a commercially available peroxide rinse, which does not include a catalase inhibitor.

### Specific Example 4

A study was performed to examine the decomposition rate of peroxide in compositions containing the catalase inhibitors shown in Table 6 versus the commercial mouth rinse, PEROXYL®, which does not contain a catalase inhibitor. In this study 1 mL of the mouth rinse was incubated in 5 g of stimulated saliva collected from the same individual for 2 minutes. 10 mL of glacial acetic acid was then added to stop the reaction. The percentage of peroxide was then determined using the standard thiosulfate method.

The results are shown in Table 8.

TABLE 8

Percent (%) loss of
hydrogen peroxide

| | | |
|---|---|---|
| Time after incubation (minutes) | 0 | 2 |
| Commercial Rinse | 100 | 11.9 |
| Rinse (Table 6) | 100 | 5.1 |

The results in Table 8 show that the inventive composition containing the catalase inhibitor and the anti-oxidant has less degradation of peroxide compared to the commercial rinse (PEROXYL®) containing hydrogen peroxide as the active ingredient, ethyl alcohol (6% v/v), FD&C green #3, FD&C yellow #5, menthol, methyl salicylate, poloxamer 338, polysorbate 20, purified water, sodium saccharin, and sorbitol solution (70%). The greater peroxide stability in the inventive composition indicates less degradation, thus fewer free radicals.

In the compositions described above, other ingredients such as ethanol, flavor agents, agents to prevent dental hypersensitivity and other ingredients that are known to have beneficial effects in the oral cavity can be added.

### Specific Example 5

Table 9 below, illustrates an example of an alcoholic high peroxide teeth bleaching gel.

TABLE 9

| Ingredient | % w/w |
|---|---|
| CARBOPOL 940 ® | 5 |
| Water | 27.1 |
| Hydrogen peroxide (50%) | 60 |
| Ethanol (95%) | 10 |
| Phosphoric Acid (80%) | 0.2 |
| Sodium Lauryl sulfate | 0.2 |
| Sodium Pyrophosphate | 0.2 |
| Sodium Acetate | 0.2 |
| Triethyl Citrate | 0.1 |

### Specific Example 6

Table 10 below, illustrates an example of an alcoholic mouth rinse.

TABLE 10

| Ingredient | % (W/W) |
|---|---|
| Water (distilled) | 72.45 |
| Ethanol (95%) | 10 |
| Hydrogen Peroxide (35% Solution) | 9 |
| Pluronic F68 | 5 |
| Sodium Acetate (30% Solution) | 1.25 |
| Pluronic F127 | 1 |
| Phosphoric Acid (80%) | 0.4 |
| Sodium Pyrophosphate | 0.4 |
| Polysorbate 20 | 0.2 |
| Sodium Lauryl Sulfate | 0.2 |
| Flavor | 0.1 |
| Triethyl Citrate | 0.1 |
| Saccharin | 0.1 |

The invention also provides a delivery system to administer the oral composition of the invention. In an embodiment of the invention, the oral composition may be applied to teeth in the form of a gel or a mouth rinse that exhibits superior teeth whitening effects when compared to compositions that do not contain hydroperoxidase inhibitors. Furthermore, the compositions of the invention may be used in the dental office and also for in-home use, splint applications, paint on applications, and application using strips of polymeric materials.

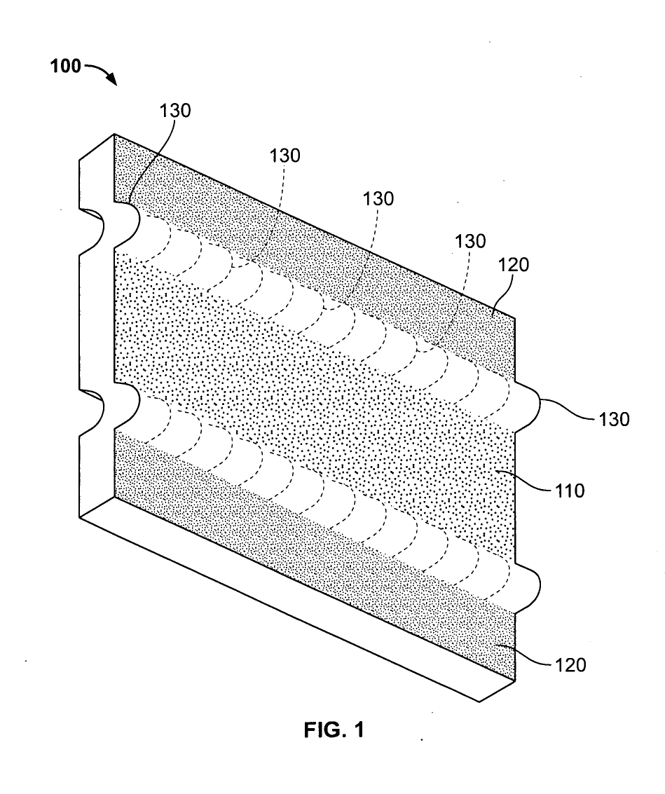

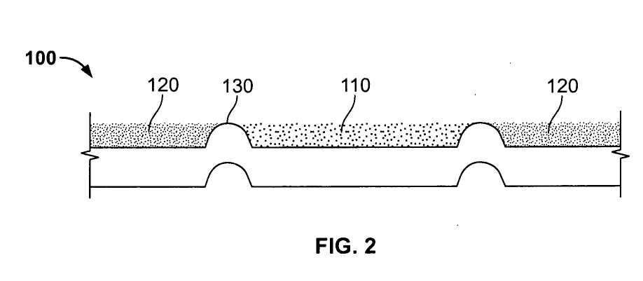

FIG. 1 is a top view of the delivery system according to an embodiment of the invention in which the oral composition is applied using a strip of polymeric material. FIG. 2 is a cross-sectional view of the delivery system of the invention as shown in FIG. 1.

As shown in FIG. 1, the delivery system comprises a flexible film 100, an oral care composition 110 that contacts the teeth, an adhesive material 120 that generally contacts the soft tissues, and a physical barrier 130 that prevents contact between the oral care composition 110 and the adhesive material 120. On the flexible film 100, the oral care composition 110 and the adhesive 120 are separated by the physical barrier 130.

The delivery system is generally less than about 5 mm thick. The length and the width may vary depending on the number and the type of teeth to be treated. For example, the delivery system may be about 15 cm long or the approximate distance from one third molar to the other third molar i.e., tooth number 1 to tooth number 16. The delivery system may also be manufactured in different lengths to suit the needs of a particular individual or to fit a particular dental arch. The length may also be adjusted or reduced by the user or the dental professional to treat specific teeth. In addition, the barrier and the adhesive material may also be placed in the molar regions to provide for extra adhesion.

The strip may be about 5 cm wide, and preferably about 2.5 cm to about 3.5 cm wide. For example, if the delivery system is about 3 cm wide, the oral care composition 110 may be about 2 cm wide and the adhesive material 120 may be about 1 cm wide i.e., 0.5 cm on the facial aspect and 0.5 cm on the lingual aspect.

The physical barrier 130 separating the peroxide preparation may be less than about 0.5 cm thick. Preferably the thickness of the physical barrier is generally less than about 3 mm thick. For example, the physical barrier 130 may be an indentation or a raised region on the flexible film 100 as shown in FIG. 1 and FIG. 2. The physical barrier 130 may be formed continuously along the flexible film 100, or it may be formed in a segmented manner.

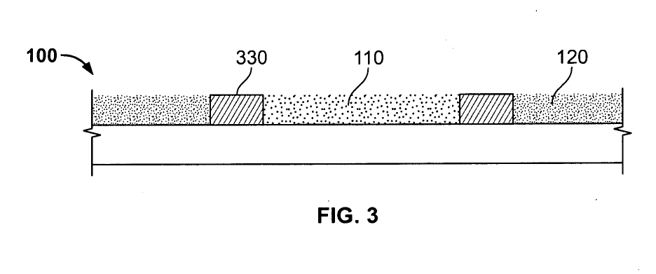

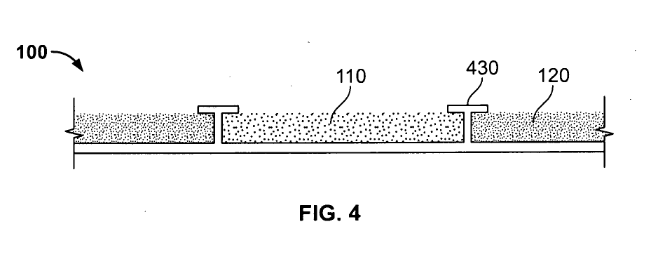

FIG. 3 and FIG. 4 are cross-sectional views according to other embodiments of the invention.

The physical barrier may also include dividing strips 330 that are placed or bonded on the flexible film as illustrated in FIG. 3. The barrier may assume any suitable shape to prevent the mixing and contact of the adhesive material 320 with oral composition 310 that is applied to the surfaces to the teeth. An example of a “T” shaped physical barrier 430 is illustrated in FIG. 4, but the shape of the physical barrier is not limited thereto.

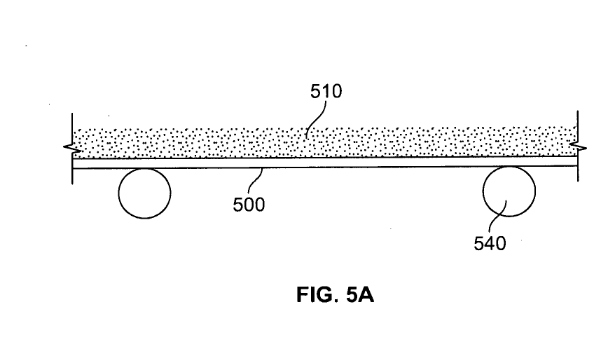

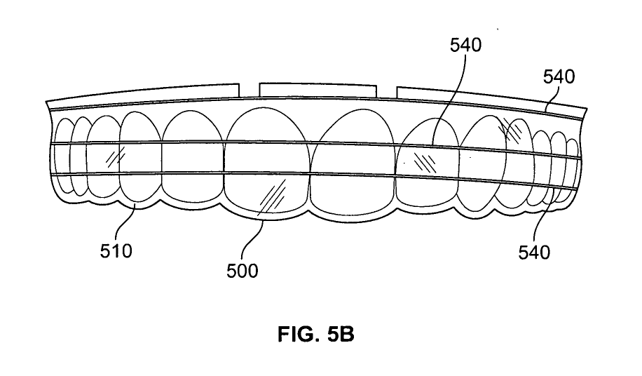

FIG. 5A is a cross-sectional view of the delivery system according to another embodiment of the invention. FIG. 5B is a front view of the delivery system shown in FIG. 5A as applied to teeth.

As shown in FIG. 5A and FIG. 5B, the delivery system includes a flexible film 500, an oral care composition 510, and orthodontic wire 540 that runs along the length of the flexible film 500. A more rigid delivery system may be prepared by adding the orthodontic wire 540 at regular intervals such as at 0.1 cm to about 1 cm intervals.

The orthodontic wire 540 is used to increase the mechanical retention of the strip, prevent its deformation, and increase the rigidity of the strip, thus making it possible to exclude the adhesive material. Other flexible materials may be used instead of the copper wire such as metal wires including stainless steel wires and aluminum wires; polymeric wires; and thermal arch wires such as TRUFLEX® and ORMCO®. The advantages of using thermal arch wires are that they have a low degree of angular deflection at lower temperatures and, thus, require less force to shape at low temperatures. At body temperature, they require greater forces to shape them and thus they retain their shape better and resist dislodgement of the strip.

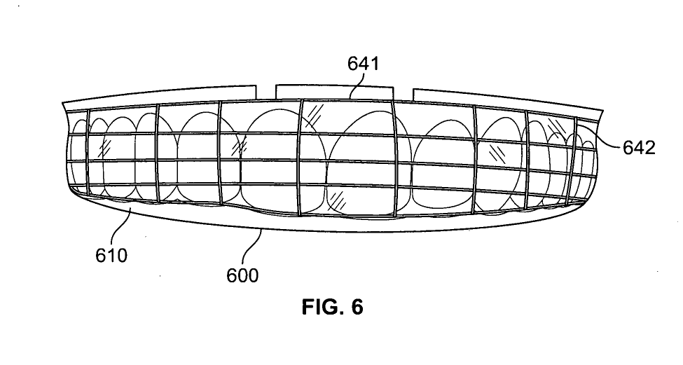

FIG. 6 is a front view of a delivery system according to another embodiment of the invention, as applied to teeth.

As shown in FIG. 6, the delivery system includes a flexible film 600, an oral care composition 610, and a scaffold that is formed using horizontal orthodontic wire 641 and vertical orthodontic wire 642. The scaffold may be made with inserts in any combination as long as the delivery system resists dislodgment.

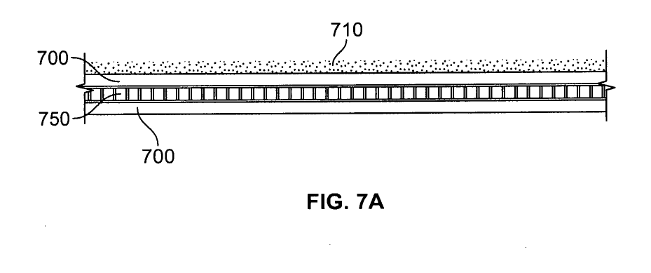

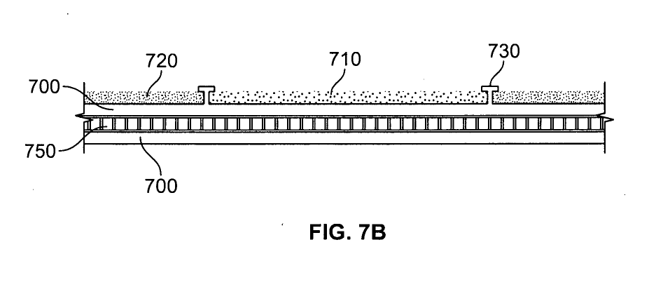

FIG. 7A and FIG. 7B are cross-sectional views of the delivery system according to other embodiments of the invention. As shown in FIG. 7A and FIG. 7B, the delivery system includes two flexible films 700, an oral care composition 710, an adhesive 720, and a wire mesh 750. FIG. 7B additionally shows the delivery system to include physical barriers 730.

The wire mesh 750 may comprise an aluminum screening material such as VISTA-WEAVE® (Phifer Wire Products Inc.), but is not limited thereto. Similar mesh materials made from metallic alloys, fiberglass, and other polymeric materials may also be used.

It is well known that metals may react with materials such as peroxide, thus a polymeric material may be used in the mesh to avoid such interactions. The delivery system including the mesh 750 may be prepared by thermally bonding the mesh or using adhesives to attach it to the flexible films 700 to form a laminate. The laminate has the advantage that dyes, colorants, fluorescent, phosphorescent and/or chemiluminescent materials may be included within the laminate. The mesh may be about 0.005 mm to about 7 mm thick and, preferably, from about 0.05 to about 1 mm thick.

Metals and metallic alloys may also be used in bleaching of teeth because they can be used as electrodes to enable the movement of peroxide, electrophoretically remove dental stain and/or apply heat to the teeth and the whitening composition. For example, a battery can be connected with the negative terminal connected to the facial surface and the positive terminal on the lingual side. Thus, the current travels from one surface of the teeth to the other through the whitening composition.

The flexible film may comprise the materials described in U.S. Pat. No. 6,096,328, which is incorporated herein by reference. In particular, the flexible film may comprise polymers, natural and synthetic woven materials, non-woven material, foil, paper, rubber, and combinations thereof. Examples of suitable materials may include, but are not limited to, polyethylene, ethylvinylacetate, polyesters, ethylvinyl alcohol and combinations thereof. Examples of polyesters may include biaxially-oriented polyethylene terephthalate (such as MYLAR®) and fluoroplastics including polytetrafluouroethylene (such as TEFLON®). The flexible film may be less than about 3 mm thick and, preferably, is about 0.001 mm to about 1 mm thick.

The oral care composition may include the oral composition of the invention, described earlier, in addition to other workable compositions.

The adhesive material may comprise a water-soluble hydrophilic colloid or polymer having the property of swelling upon exposure to moisture to form a mucilaginous mass. Such adhesive materials include natural gums, synthetic polymeric gums, alkyl vinyl ether-maleic acid copolymers such as GANTREZ® (GAF Corporation), synthetic polymers, mucoadhesive polymers, hydrophilic polymers, saccharide derivatives, cellulose derivatives, and adhesive materials commonly employed in denture stabilizing compositions and compatible with the subject polymers of the present invention, and mixtures thereof. Examples of such materials may include, but are not limited to, karaya gum, guar gum, gelatin, algin, sodium alginate, tragacanth, chitosan, polyethylene glycol, acrylamide polymers, carboxypolymethylene (such as CARBOPOL®), polyvinyl alcohol, polyamines, polyquartemary compounds, polybutenes, silicones, silicon resins, silicon gums ethylene oxide polymers, polyvinylpyrrolidone, cationic polyacrylamide polymers. In addition, the materials described in U.S. Patent Application No. 20050019276 such as VASELINE® (commercially available from Cheeseborough Ponds) or denture adhesive such as FIXODENT® (commercially available from Warner Lambert Inc.) may be used.

The adhesive material may include other adhesive materials used in bleaching of teeth. These include barrier materials which polymerize in presence of a curing light. The adhesive material may also include any ingredient that counteracts the damaging effects of peroxide. These ingredients include inhibitors of Nuclear Factor-kappa B (NF-kB) which is known to be over expressed in conditions of oxidative stress, certain cancers such as oral cancer and induced by hydrogen peroxide (Howe et al., Journal of Biological Chemistry, Volume 277, 2002, pages 30469-30476). A list of inhibitors useful for this invention has been published by Pande and Ramos in Current Medicinal Chemistry, 2005, Volume 12, pages 357-374. The inhibitors may be used individually or in any combination. Preferable inhibitors of NF-kB include compounds with a known safety profile. More preferable inhibitors of NF-kB include compounds such as alpha lipoic acid, curcumin, dihydro lipoic acid, dimethyl sulfoxide, glutathione, vitamin E like antioxidants, melatonin, sodium salicylate, ibuprofen, hydroxyqunione and omega-3 fatty acids, esters of omega-3 fatty acids, individually or in any combination. These inhibitors are also especially useful with smokers and users of tobacco who desire whiter teeth because tumor promoters present in tobacco are known to induce NF-kB.

Studies have also shown that cancer cell migration and adhesion occurs through a hydrogen peroxide mediated mechanism (Payne et al, Cancer Res, 65:11429-11436, 2005). The adhesion and migration is thought to occur when the basement membrane is degraded by proteases. Further, protease inhibitors, such as alpha-1-trypsin inhibitor are readily inactivated by oxidizing agents (Nathoo and Finlay, Arch. Biochem. Biophphys. 246:162-174, 1986). Thus it is desirable to include protease inhibitors within the adhesive barrier material. The protease inhibitors include inhibitors of calcium dependent proteases, inhibitors of metallo-proteases, inhibitors of serine proteases, the inhibitors of threonine proteases, inhibitors of aspartic acid proteases and inhibitors of glutamic acid proteases. The preferred inhibitors are inhibitors of serine proteases, their analogs and homologs, including therapeutic preparations such as Trasylol® (commercially available from Bayer). These inhibitors include Kunitz and Kazal inhibitors. The most preferred inhibitors include the Bowman-Birk, the soya bean trypsin inhibitor and therapeutic preparations such as Trasylol® (commercially available from Bayer) which are used to control inflammation.

The use of the Bowman-Birk inhibitor to suppress and inhibit carcinogenesis has been disclosed in U.S. Pat. Nos. 5,338,547 and 5,616,198. In these disclosures, the inhibitor can be administered in the form of a mouth wash or a saliva substitute. Disadvantages of this type of delivery system is that they have a limited residence time in the oral cavity when compared to an adhesive oral composition. Notably, a delivery system containing film forming polymers as described in U.S. Patent Application number 20060062743 can be used to increase the residence time. Further, U.S. Pat. Nos. 5,338,547 and 5,616,198 do not disclose the use of other ingredients such as inhibitors of NF-kB in combination with the protease inhibitors. As indicated above, serine protease inhibitors such as alpha-1-trypsin inhibitor is susceptible to oxidation therefore inclusion of anti-oxidants within the composition are also useful.

As indicated previously, bleaching agents are known to release mercury from dental amalgam. Along with oxidation and UV damage, some heavy metals have been shown to damage DNA, leading to alteration in the tumor suppressor p53 protein (Tassabehji et al., Exp Biol Med, 230:699-708, 2005). Hence compounds which interact with mercury and/or uv light can also be added to the adhesive material. Such compounds include various chelating agents which form irreversible complexes with mercury and other compounds such as selenium, amino-acid selenium compounds such as methionine—selenium, sodium selenite and the like. Other useful ingredients include activated carbon and sodium bicarbonate. The preferable levels of activated carbon are in the range of about 0.01% to about 10% by weight of the composition. More preferably, the levels of activated carbon range from about 0.1% to about 5% by weight of the composition.

Other compounds that can be included in the adhesive material include, for example, anti-microbial materials such as triclosan and other phenolic anti-microbial agents, vitamins such as vitamin E and its analogs, and natural ingredients such as aloe vera, ubiquinone may be used. Other anti-oxidants such as vitamin C and its analogs may also be included in the adhesive component to further aid in prevent damage from oxygen radicals. Other useful agents such as zinc compounds for preventing halitosis may also be included. The adhesive may also include agents such as potassium nitrate to prevent dental hypersensitivity. Other materials such as fluorescent, phosphorescent or chemiluminescent materials, such as those described in U.S. Patent Application Number 20050026103, may also be included in the adhesive material.

Other possible additives to the adhesive material include, but are not limited to, colorants, preservatives such as methyl and propyl parabens, thickeners such as silicon dioxide, and polyethylene glycol, and vehicles such as liquid petrolatum, petrolatum, mineral oil, and glycerin. Colorants, flavors, sweetners, preservatives, thickeners and vehicles may have a concentration of from about 0% to about 20%, by weight of the composition.

Examples of useful FIXODENT® type adhesive composition are shown below:

| Ingredient | % w/w |
|---|---|
| Petrolatum | 32.28 |
| Mineral Oil | 14 |
| D&C Red | 0.02 |
| Hydrated Silica | 2.0 |
| Gantrez MS-955 | 33 |
| Cellulose Gum | 19 |
| Sodium Saccharin | 0.1 |
| Triethyl Citrate | 0.2 |
| Curcumin | 0.1 |
| Selenium | 0.00001 |
| Omega-3 fatty acid | 0.2 |

The physical barrier may comprise any polymeric material that does not react with the adhesive material and the oral composition. For example, the physical barrier may be comprised of polymers, natural and synthetic woven materials, non-woven material, foil, paper, rubber, metal, alloys and combinations thereof. Examples of these include, but are not limited to, polyethylene, ethylvinylacetate, polyesters, ethylvinyl alcohol and combinations thereof. Examples of polyesters include biaxially-oriented polyethylene terephthalate (such as MYLAR®) and fluoroplastics, including polytetrafluouroethylene (such as TEFLON®).

The invention has been disclosed broadly and illustrated in reference to representative embodiments described above. Those skilled in the art will recognize that various modifications can be made to the invention without departing from the spirit and scope thereof.

Without further elaboration, it is believed that one skilled in the art, using the preceding description, can utilize the invention to the fullest extent. The following examples are illustrative only, and not limiting of the disclosure in any way whatsoever.

### EXAMPLES

### Example 1

### Physical Barrier is Tubing

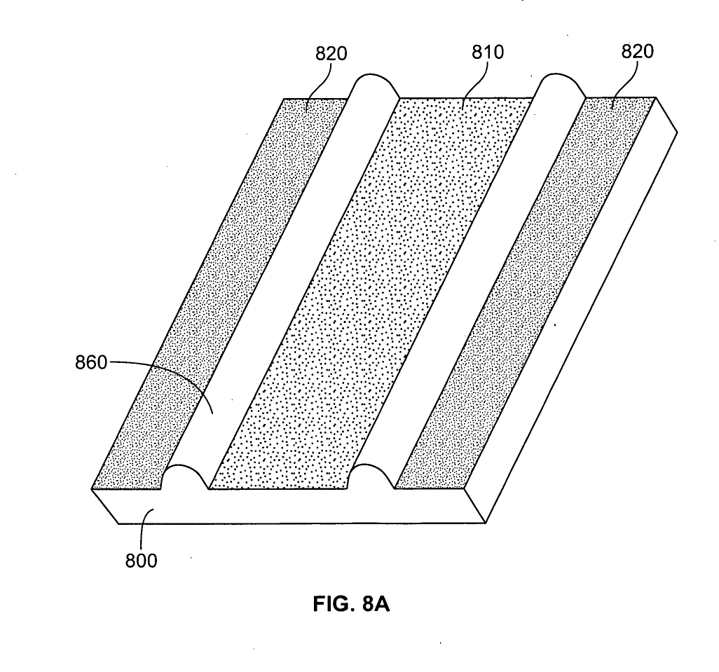

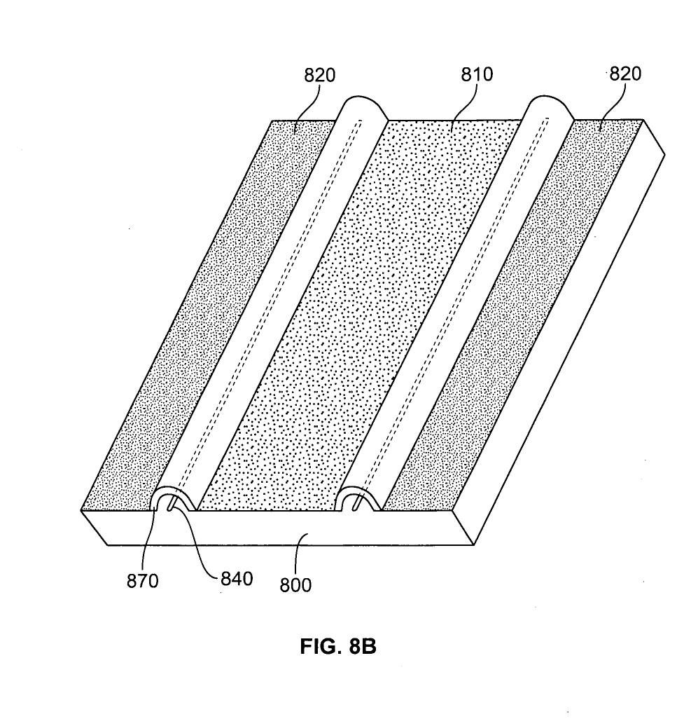

FIG. 8A is a top view of a delivery system according to another embodiment of the invention, as described in Example 1. As shown in FIG. 8A, a 15 cm long by 3 cm wide polyethylene strip was prepared as a flexible film 800 by cutting a ZIPLOC® bag (commercially available from S.C. Johnson & Sons). The polyethylene strip 800 was then divided into three sections and orthodontic plastic tie tubing 860 (approximately 0.6 mm, outer diameter) was bonded along the length of the strip using cyanoacrylate glue at approximately 1 cm from both edges.

In this case, the orthodontic plastic tie tubing 860 acted as a physical barrier. The hydrogel peroxide preparation as the oral care composition 810 as described in U.S. Pat. No. 6,746,679 was placed in the center and FIXODENT® Denture Adhesive Cream as adhesive material 820 (commercially available from Proctor & Gamble) was placed at either end. Contact between the oral care composition 810 and the adhesive 820 was prevented by the physical barrier 860.

### Example 2

### Physical Barrier is Plastic Sleeve with Wire Inserted Therein

FIG. 8B is a top view of a delivery system according to another embodiment of the invention, as described in Example 2.

As shown in FIG. 8B, the flexible film 800 was made out of the ZIPLOC® bag that was cut as described above. Then, instead of using orthodontic plastic tie tubing as in Example 1, orthodontic arch wire sleeve 870 with an inner diameter of about 0.8 mm and an outer diameter of 1.6 mm was adhered to the strip as described above. Then, an orthodontic wire 840, such as a copper wire, was inserted into the plastic arch wire sleeve to increase the mechanical retention of the strip, prevent its deformation, and increase the rigidity of the strip, thus making it possible to exclude the adhesive material.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in the chemical and nanotechnology fields or related fields are intended to be within the scope of the appended claims.

Without departing from the spirit of the invention, oxidative stress in the oral cavity or any bodily structure, for example, skin, gastro-intestinal system, cardio-vascular system, or respiratory system in humans and animals can also be reduced by using the above mentioned ingredients such as, but not limited to, organic acids like ascorbic acid, salicyclic acid, compounds of salicyclic acid such as acetyl salicyclic acid and alike, adipic acid, tartaric acid, omega 3 fatty acids, esters of omega 3-fatty acids, unsaturated fatty acids ubiquniones such as Coenzyme Q, sorbic acid, amines such as lecithin, or amino acids such as glutamine, methionine and cysteine, or esters such as ascorbil palmitate, ascorbil stearate and triethylcitrate, other compounds such as selenium, amino-acid selenium compounds such as methionine—selenium and the like, sodium selenite, and inhibitors of NF-kB in tooth pastes, tooth gels, mouth rinses, flosses, chewing gums, candies, lozenges, paint-on products, prophy pastes, tablets, sprays, soft drinks, skin care preparations, first aid preparations, denture adhesives, cavity cleansers, cavity, lines, cavity bases and the like. The ingredients described may also be used within dental restorative materials. For example a safe level of selenium or its compounds can be included within dental amalgams and/or can be placed in cavities, for example, as liners or bases before placing amalgam. Other useful ingredients include activated carbon and sodium bicarbonate. The preferable levels of activated carbon are in the range of about 0.01% to about 10% by weight of the composition. More preferably, the levels of activated carbon range from about 0.1% to about 5% by weight of the composition.

To reduce oxidative stress and prevent damage to the oral cavity, the oral care compositions can be used to maintain regular oral hygiene or can be used before and/or after using peroxide containing products, for example, before and/or after teeth bleaching in the dental office and/or at home. Representative examples of tooth pastes, mouth washes, etc. are presented below but can be added to any commercial oral care product or oral care formulation, including dental filling materials such as composites, glass-ionomers, adhesives, bonding materials, impression materials, amalgams, etching materials, tooth conditioning materials, periodontal gels, topical anesthetics, cavity liners, cavity bases, carie detectors, endodontic irrigating solutions, endodontic filling materials, denture bases, and the like.

Further, selenium and selenium compounds may also be added to dental amalgams to control the release of mercury.

Tooth paste to reduce oxidative stress and metal toxicity

| Ingredient | % wt |
|---|---|
| sorbitol (70% Solution) | 55.15 |
| Abrasive Silica (Syloid ® 74) | 13.00 |
| Glycerol | 11.80 |
| PEG 300 | 5.00 |
| Triethly Citrate | 1.00 |
| Thickening Silica (Syloid ® 63) | 3.00 |
| Flavor | 2.00 |
| Sodium Lauryl Sulfate | 2.0 |
| Tri-sodium phosphate | 1.5 |
| Sodium saccharin | 1.00 |
| Omega 3 fatty acid | 1.00 |
| Xanthum gum | 0.60 |
| Bowman-Birk Inhibitor | 0.5 |
| CARBOPOL 940 | 0.30 |
| sodium fluoride | 0.24 |
| Curcumin | 0.05 |
| Selenium | 100 micro grams |
| Water | to 100% |

The pH of the above composition may be adjusted to between 6 and 10, preferably to about 8.

Tooth paste compositions can also be substantially anhydrous whereby aqueous sorbitol and water are replaced by other carriers such as ethanol, glycerol, polyethylene glycol, propylene glycol and the like. Pigments and dyes such as titanium dioxide, FD&C food colorants can also be added to provide the desired color and disguise the yellow color of curcumin.

Mouth Rinse to reduce oxidative stress and metal toxicity

| Ingredient | % wt |
|---|---|
| Glycerol | 15.00 |
| Ethyl Alcohol | 15.00 |
| Polysorbate-80 | 1.0 |
| Xylitol | 1.0 |
| Omega 3 fatty acid | 0.5 |
| Triethyl citrate | 0.5 |
| Potassium sorbate | 0.15 |
| Curcumin | 0.01 |
| Flavor | 0.1 |
| Selenium | 100 micrograms |
| Water | to 100% |

The pH of the mouth rinse can be adjusted to between 7 and 11, preferably to about 8.5.

The above compositions can be manufactured by standard methods with the exception that curcumin is first mixed with omega 3 fatty acid, this is followed by the addition of alcohol and flavor to dissolve the curcumin. The color of the composition can be adjusted by addition of dyes and colorants if desired.

To examine the effects of NF-kb and selenium upon peroxide bleaching of teeth the following composition was prepared.

| Ingredient | % (w/w) |
|---|---|
| Ethanol | 9 |
| Pluronic F68 | 5 |
| Sodium Acetate (30% Solution) | 1.25 |
| Pluronic F127 | 1 |
| Sodium Pyrophosphate | 0.4 |
| Omega 3 fatty acid | 0.25 |
| Polysorbate 20 | 0.2 |
| Sodium Lauryl Sulfate | 0.2 |
| Triethyl Citrate | 0.1 |
| Elemental Selenium | 5 micrograms |
| Distilled water to 100% | |

The effects of the above rinse was examined by using six extracted human molars. In this study the color of the teeth was measured using a standard dental shade guide (Vita®). After extraction and the teeth were divided into two groups. One group of teeth was soaked in distilled water for one minute, while the other group was soaked in the above solution. The teeth were then transferred to a 3% hydrogen peroxide solution, allowed to bleach for one hour, and the color was measured again after bleaching. The change in the color of the teeth was then calculated as described previously (Nathoo et al, Compend. Contin Edu. Dent. S17:S640-S645, 1994). The results of this study are presented below and show that the above rinse did not interfere with the bleaching process and may aid in the bleaching process.

Vita ® Shade

| Sample | Baseline | After Soaking | After Peroxide Treatment |
|---|---|---|---|
| Control | 7.3 | 7.3 | 8.4 |
| Inventive Comp. | 7.1 | 7.3 | 9.00 |

Toothpaste to reduce oxidative stress and metal toxicity

| Ingredient | % wt |
|---|---|
| Sorbitol (70% solution) | 55.15 |
| Abrasive Silica (Syloid ® 74) | 13.00 |
| Glycerol | 11.80 |
| PEG 300 | 5.00 |
| Triethyl Citrate | 1.00 |
| Thickening Silica (Syloid ® 63) | 3.00 |
| Flavor | 2.00 |
| Sodium Lauryl Suldate | 2.00 |
| Sodium Bicarbonate | 1.50 |
| Sodium Saccharin | 1.00 |
| Titanium Dioxide | 1.00 |
| Xanthum gum | 0.60 |
| Activated carbon | 0.50 |
| CARBOPOL 940 | 0.30 |
| Sodium Fluoride | 0.24 |
| Sodium Selenite | 0.01 |
| Water | to 100% |

The above composition may be used to contact the teeth in any manner before, after and/or during the bleaching process. If desired, a peroxide compound, additional thickening agents and carrier materials can also be incorporated. Elemental selenium can also be replaced with compounds of selenium such as selenocystine and selenomethionine. Inhibitors of NF-Kb can also be replaced with other compounds described above and the pH adjusted to between about 3 to about 11, preferably to between about 5 to about 10, more preferably to about 8.

The disclosures of all references and publications cited above are expressly incorporated by reference in their entireties to the same extent as if each were incorporated by reference individually.

What is claimed is:

1. A method for preparing an oral care composition, comprising:
providing a flexible film;
applying an oral care composition to the flexible film;
applying an adhesive material to the flexible film; and
forming a physical barrier that prevents contact between the oral composition and the adhesive material.

2. The method of claim 1, wherein the flexible film is less than about 3 mm thick.

3. The method of claim 2, wherein the flexible film includes at least one of polymers, natural and synthetic woven materials, non-woven material, foil, paper, rubber, and combinations thereof.

4. The method of claim 1, wherein the oral care composition includes a teeth bleaching agent in an orally acceptable vehicle.

5. The method of claim 1, wherein the oral care composition includes:
a peroxide compound;
a hydroperoxidase inhibitor;
a chelating agent;
an anti-oxidizing agent;
a thickening agent; and
an orally acceptable vehicle.

6. The method of claim 1, wherein the adhesive material includes a hydrophilic or a hydrophobic bioadhesive material.

7. The method of claim 1, wherein the adhesive material includes at least one of natural gums, synthetic polymeric gums, alkyl vinyl ether-maleic acid copolymers, synthetic polymers, mucoadhesive polymers, hydrophilic polymers, saccharide derivatives, cellulose derivatives, and adhesive materials commonly employed in denture stabilizing compositions, and combinations thereof, and
wherein the adhesive material is compatible with the oral care composition and the flexible film.

8. The method of claim 1, wherein the physical barrier is an indentation or a raised region of the flexible film, a dividing strip that is placed or bonded to the flexible film, a T-shaped barrier, an orthodontic tube, or an orthodontic sleeve with a wire inserted in the orthodontic sleeve.

9. The method of claim 8, wherein the wire includes metal wires including copper wire, stainless steel wires, and aluminum wires, polymeric wires, and thermal arch wires.

10. The method of claim 1, further comprising forming an orthodontic wire along a length of the flexible film.

11. The method of claim 1, further comprising forming an orthodontic wire along a length and a width of the flexible film.

12. The method of claim 1, further comprising forming a laminate including a mesh formed in between two flexible films.

13. The method of claim 12, wherein the physical barrier may be an indentation or a raised region of the flexible film, a dividing strip that is placed or bonded to the flexible film, a T-shaped barrier, an orthodontic tube, or an orthodontic sleeve with an orthodontic wire inserted in the orthodontic sleeve.

14. A delivery system for an oral care composition, comprising:
a flexible film;
an oral care composition to the flexible film;
an adhesive material to the flexible film; and
a physical barrier that prevents contact between the oral composition and the adhesive material.

15. The delivery system of claim 14, wherein the flexible film is less than about 3 mm thick.

16. The delivery system of claim 15, wherein the flexible film is selected from the group consisting of polymers, natural and synthetic woven materials, non-woven material, foil, paper, and rubber and combinations thereof.

17. The delivery system of claim 14, wherein the oral care composition comprises a teeth bleaching compound in an orally acceptable vehicle.

18. The delivery system of claim 14, wherein the oral care composition includes:
a peroxide compound;
a hydroperoxidase inhibitor;
a chelating agent;
an anti-oxidizing agent;
a thickening agent; and
an orally acceptable vehicle.

19. The delivery system of claim 14, wherein the adhesive material comprises at least one of natural gums, synthetic polymeric gums, alkyl vinyl ether-maleic acid copolymers, synthetic polymers, mucoadhesive polymers, hydrophilic polymers, saccharide derivatives, cellulose derivatives, and adhesive materials commonly employed in denture stabilizing compositions, and combinations thereof, wherein the adhesive material is compatible with the oral care composition and the flexible film.

20. The delivery system of claim 19, wherein the adhesive material further comprises at least one of an effective amount of anti-oxidant, an effective amount of an inhibitor of Nuclear Factor-kB (NF-kB), and an effective amount of sequesterant of heavy metals, and combinations thereof.

21. The delivery system of claim 20, wherein the anti-oxidizing agent includes at least one of triethyl citrate, omega 3-fatty acids, and selenium, and mixtures thereof.

22. The delivery system of claim 20, wherein the Nuclear Factor-kB inhibitor is selected from the group consisting of curcumin, alpha lipoic acid, salcyclic acid, salts of salcyclic acid, melatonin and dihydrolipoic acid, and mixtures thereof.

23. The delivery system claim 20, wherein the sequesterant includes at least one of elemental selenium and selenium compounds or combinations thereof.

24. The delivery system of claim 14, wherein the physical barrier is an indentation or a raised region of the flexible film, dividing strips that are placed or bonded to the flexible film, a T-shaped barrier, an orthodontic tube, or an orthodontic sleeve with a wire inserted in the orthodontic sleeve.

25. The delivery system of claim 24, wherein the wire includes metal wires including copper wire, stainless steel wires, and aluminum wires, polymeric wires, and thermal arch wires.

26. The delivery system of claim 24, further comprising forming an orthodontic wire along a length of the flexible film.

27. The delivery system of claim 24, further comprising forming an orthodontic wire along a length and a width of the flexible film.

28. The delivery system of claim 24, further comprising forming a laminate including a mesh formed in between two flexible films.

29. The delivery system of claim 28, wherein the physical barrier is an indentation or a raised region of the flexible film, dividing strips that are placed or bonded to the flexible film, a T-shaped barrier, an orthodontic tube, or an orthodontic sleeve with a wire inserted in the orthodontic sleeve.

30. A method for reducing oxidative stress induced by applying oxidizing agents to a tooth comprising the step of:
applying to the teeth a composition including at least one of an inhibitor of Nuclear Factor-kB, an anti-oxidizing agent, and a metal sequesterant, and combinations thereof.

31. A composition for reducing oxidative stress induced by oxidizing agents comprising, at least one of an inhibitor of Nuclear Factor-kB, an anti-oxidizing agent, activated carbon, and a metal sequesterant, and combinations thereof.

32. The composition of claim 31, wherein the effective amount of the inhibitors of Nuclear Factor-kB is at least about 0.01 percent by weight.

33. The composition of claim 32 wherein the inhibitors of Nuclear Factor-kB are selected from the group consisting of curcumin, alpha lipoic acid, salicylic acid, salts of salicylic acid, melatonin and dihydrolipoic acid, and mixtures thereof.

34. The composition of claim 31, wherein the effective amount of the anti-oxidizing agent is at least about 0.01 percent by weight.

35. The composition of claim 34, wherein the anti-oxidizing agent is selected from the group consisting of triethyl citrate, omega 3-fatty acids, and their esters and mixtures thereof.

36. The composition of claim 20, wherein the effective amount of metal sequesterant is at least about 0.1 microgram.

37. The composition of claim 36, wherein the metal sequesterant is a source of selenium selected from the group consisting of elemental selenium, selenomethionine and selenocysteine, and mixtures thereof.

38. The composition of claim 31, further comprising an orally acceptable vehicle including at least one of carriers or flavorings suitable for intra-oral administration.

39. The composition of claim 31, wherein the activated carbon is present in an amount ranging from about 0.01% to about 5% by weight of the composition.

40. A composition for reducing oxidative stress induced by teeth whitening agents comprising:
an orally acceptable vehicle; and
at least one of an effective amount of an inhibitor of Nuclear Factor-kB, an effective amount of an anti-oxidizing agent, and an effective amount of a metal sequesterant, and combinations thereof.

41. The composition of claim 40, wherein the effective amount of inhibitor of Nuclear Factor-kB is at least about 0.01 percent by weight.

42. The composition of claim 41, wherein the inhibitor of Nuclear Factor-kB is selected from the group consisting of curcumin, alpha lipoic acid, salicylic acid, salts of salicylic acid, melatonin, dihydrolipoic acid, omega 3 fatty acids, and their esters, and combinations thereof.

43. The composition of claim 40, wherein the effective amount of the anti-oxidizing agent is at least about 0.01 percent by weight.

44. The composition of claim 43, wherein the anti-oxidizing agent is selected from the group consisting of triethyl citrate, omega 3-fatty acids, and their esters, and combinations thereof.

45. The composition of claim 40, wherein the effective amount of the metal sequesterant is at least about 0.1 microgram.

46. The composition of claim 45, wherein the metal sequesterant is a source of selenium selected from the group consisting of elemental selenium, selenomethionine, selenocysteine, and sodium selenite and combinations thereof.

47. The composition of claim 40, further comprising at least one of a carrier and a flavoring suitable for intra-oral administration.

48. The composition of claim 40, further comprising activated carbon present in an amount ranging from about 0.01% to about 5% by weight of the composition.

49. A method for reducing oxidative stress comprising the step of:
administering an effective amount of a composition including at least one of an inhibitor of Nuclear Factor-kB, an anti-oxidizing agent, and a metal sequesterant in a suitable carrier.

50. The method of claim 49, further comprising the step of:
contacting the structures of the mouth and oral cavity with the composition.

51. The method of claim 50, wherein said contacting step may occur prior to teeth whitening.

52. The method of claim 50, wherein said contacting step may occur during teeth whitening.

53. The method of claim 50, wherein said contacting step may occur after teeth whitening.

54. The method of claim 50, wherein the composition further comprises a tooth whitening agent.

55. The method of claim 54, wherein the composition is administered orally in the form of a tablet or lozenge to reduce oxidative stress occurring as a result of pulpal penetration of the teeth whitening agent.
